(19) United States
(12) Patent Application Publication
(10) Pub. No.: US 2022/0034810 A1
(43) Pub. Date: Feb. 3, 2022

(54) DEVICE AND METHOD FOR AN INTRAOPERATIVE CANCER DETECTOR

(71) Applicant: ORFACTORY, Inc., Great Neck, NY (US)

(72) Inventor: David Jaehyun Park, Beachwood, OH (US)

(73) Assignee: ORFACTORY, Inc., Great Neck, NY (US)

(21) Appl. No.: 17/343,896

(22) Filed: Jun. 10, 2021

Related U.S. Application Data
(60) Provisional application No. 63/058,637, filed on Jul. 30, 2020.

(51) Int. Cl. G01N 21/64; A61B 10/02; G01N 33/50

(52) U.S. Cl. CPC G01N 21/6428; G01N 2021/6439; G01N 33/5091; A61B 10/0283

(57) ABSTRACT

A device for intraoperative cancer detection includes an excitation fiber optic configured to excite a biological sample as a function of an intrinsic excitation wavelength, an emission fiber optic configured to detect an intrinsic emission of the biological sample, a tissue scanner module including a display window configured to visualize the intrinsic emission of the biological sample, wherein visualizing further comprises receiving a signal from a tissue scanner representing an intrinsic emission of the biological sample, and relaying a visual recording as a function of the signal to the display window, and a vacuum-line tip configured to remove a portion of the biological sample as a function of the visualized intrinsic emission of the biological sample and a haptic feedback controller.

### CROSS-REFERENCE TO RELATED APPLICATIONS

This application claims the benefit of priority of U.S. Provisional Patent Application Ser. No. 63/058,637, filed on Jul. 30, 2020, and titled “A METHOD OF AND DEVICE FOR AN INTRAOPERATIVE CANCER DETECTOR,” which is incorporated by reference herein in its entirety.

### FIELD OF THE INVENTION

The present invention generally relates to the field of cancer biology. In particular, the present invention is directed to a device for a device and method for an intraoperative cancer detector.

### BACKGROUND

Reducing the tumor burden through surgical resection remains the first step in effective tumor management, where mounting evidence has proved that surgical resection is associated with a survival benefit. However, complete tumor resection is challenging due to the diffuse distribution of tumor cells in which the tumor margins are oftentimes indistinguishable from the surrounding normal tissue under visual inspection through conventional surgical microscope.

### SUMMARY OF THE DISCLOSURE

A device for intraoperative cancer detection, the device including an excitation fiber optic configured to excite a biological sample as a function of an intrinsic excitation wavelength, an emission fiber optic configured to detect an intrinsic emission of the biological sample, a tissue scanner configured to discern a signal representing the intrinsic emission of the biological sample, a tissue scanner module including a display window configured to visualize the intrinsic emission of the biological sample, wherein visualizing further comprises receiving the signal from the tissue scanner, and relaying a visual recording as a function of the signal to the display window, and a vacuum-line tip configured to remove a portion of the biological sample as a function of the visualized intrinsic emission of the biological sample and a haptic feedback controller.

A method for intraoperative cancer detection, the method including exciting, as a function of an excitation fiber optic, a biological sample as a function of an intrinsic excitation wavelength, detecting, as a function of an emission fiber optic, an intrinsic emission of the biological sample, visualizing, as a function of a tissue scanner module including a display window, the intrinsic emission of the biological sample, wherein visualizing further comprises receiving a signal from a tissue scanner representing an intrinsic emission of the biological sample, and relaying a visual recording as a function of the signal to the display window, and removing, as a function of a vacuum-line tip, a portion of the biological sample as a function of the visualized intrinsic emission of the biological sample and a haptic feedback controller.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a block diagram of an exemplary embodiment of a device for intraoperative cancer detection;

FIG. 2 is an exemplary embodiment of a diagrammatic representation of a device for intraoperative cancer detection;

FIG. 3 is an exemplary embodiment of an isometric cross-section view illustrating a tip;

FIG. 4 is an exemplary embodiment of a diagrammatic representation of a suction tip;

FIG. 5 is a flow diagram illustrating a method of intraoperative cancer detection, according to embodiments; and

FIG. 6 is a block diagram of a computing system that can be used to implement any one or more of the methodologies disclosed herein and any one or more portions thereof.

The drawings are not necessarily to scale and may be illustrated by phantom lines, diagrammatic representations, and fragmentary views. In certain instances, details that are not necessary for an understanding of the embodiments or that render other details difficult to perceive may have been omitted.

### DETAILED DESCRIPTION

At a high level, aspects of the present disclosure are directed to a device and method for intraoperative cancer detection. In an embodiment, a fluorophore may be applied to a biological sample, wherein the intraoperative cancer detection device may be used to visualize the presence of the fluorophore and/or a fluorescent metabolite of the fluorophore in the biological sample. Fiber optics embedded in the tip of the cancer detection probe may emit light to excite the fluorophore as it is present in a biological sample. Excitation of fluorophores can be detected by emission of light via intrinsic fluorescence spectroscopy (iFS) from the fluorophores in the biological sample by a tissue scanner in the tip of the cancer detection probe. This information may be captured and relayed back to a user via the tissue scanner mounted in the tip of the intraoperative cancer detection device. Furthermore, in another embodiment and without limitation, the tip may contain a vacuum line for suction through the intraoperative cancer detection device tip to aid in removal of biological sample during surgery. In such an embodiment, an intraoperative cancer detection device combines a cancer detection tool with a suction implement affording surgeons' greater utility with a single-handed instrument where traditionally two instruments may have been used.

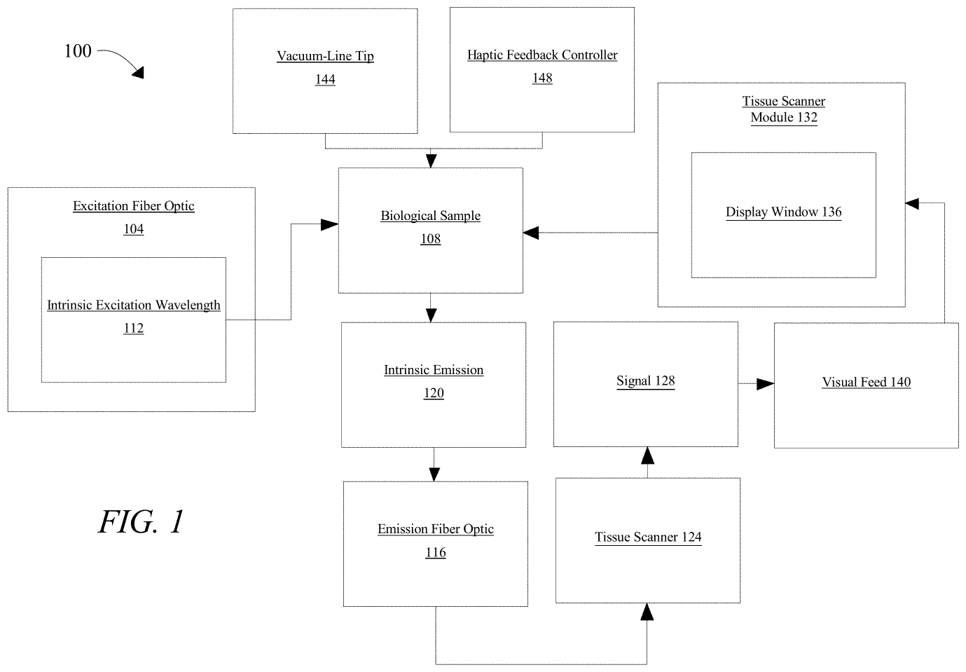

Referring now to FIG. 1, an exemplary embodiment of a device 100 for intraoperative cancer detection is illustrated. Device 100 comprises an excitation fiber optic 104. As used in this disclosure an “excitation fiber optic” is a transparent fiber that transports one or more wavelengths of light associated with an excitation wavelength. For example, and without limitation, excitation fiber optic 104 may transport a 410 nm wavelength along a transparent fiber. As a further non-limiting example, excitation fiber optic 104 may transport a 740 nm wavelength along a transparent fiber. As a further non-limiting example, excitation fiber optic 104 may transport a 280 nm wavelength along a transparent fiber. As a further non-limiting example, excitation fiber optic 104 may transport a 460 nm wavelength along a transparent fiber. As a further non-limiting example, excitation fiber optic 104 may transport a 490 nm wavelength along a transparent fiber. As a further non-limiting example, excitation fiber optic 104 may transport an 800 nm wavelength along a transparent fiber. In an embodiment, and without limitation, excitation fiber optic 104 may transport a wavelength existing in a range of wavelengths along a transparent fiber. For example, and without limitation excitation fiber optic 104 may transport a wavelength existing within the range of wavelengths of 460 nm-490 nm. As a further non-limiting example, excitation fiber optic 104 may transport a wavelength existing within the range of wavelengths of 740 nm-800 nm. Excitation fiber optic 104 may include any transparent fiber capable of transporting one or more wavelengths as a function of a total internal reflection. As used in this disclosure a “total internal reflection” is an optical phenomenon in which wavelengths of light traveling in a first medium strike against a boundary with a second medium, wherein the wavelength is reflected in the first medium with no loss of brightness. For example, and without limitation total internal reflection may include one or more critical angles, wherein the critical angle is the angle at which total internal reflection is capable, as determined by a ratio of an index of reflection of the first material to an index of refraction of the second material.

Excitation fiber optic 104 is configured to excite a biological sample 108. As used in this disclosure a “biological sample” is one or more biological components of an individual's body. Biological sample may include, without limitation cells, tissues, organs, bodily fluid, and the like thereof. As a non-limiting example, biological sample 108 may include one or more nervous tissues. As a further non-limiting example, biological sample 108 may include or more cells, tissues, and/or organs from an individual's central nervous system, peripheral nervous system, afferent division, efferent division, somatic nervous system, autonomic nervous, sympathetic division, parasympathetic division, and the like thereof. Excitation fiber optic 104 is configured to excite biological sample 108 as a function of an intrinsic excitation wavelength 112. As used in this disclosure an “intrinsic excitation wavelength” is a wavelength of light that excites a biological sample as a function of a chemical and/or fluorophore present in the biological sample. In an embodiment intrinsic excitation wavelength 112 may include a wavelength that excites a fluorophore naturally present in a biological sample such as an aromatic residue, folded protein, and the like thereof. In another embodiment, and without limitation, intrinsic excitation wavelength 112 may include a wavelength that excites a foreign fluorophore present in a biological sample such as 5-aminolevulinic acid (5-ALA), fluorescein, and the like thereof. In embodiments, excitation of biological sample 108 may include utilizing an intrinsic fluorescent probe that may be excited by an excitation wavelength. For example, and without limitation, intrinsic fluorescent probe may be configured to emit a photon at an emission wavelength as a function of being excited by an excitation wavelength, wherein the emission wavelength of the intrinsic fluorescent probe excites biological sample 108. In another embodiment, excitation fiber optic 104 may excite biological sample 108 as a function of administering an intrinsic fluorescence spectroscopy (iFS) probe to biological sample 108. For example, and without limitation, iFS probe may excite biological sample as a function of a fluorescence wavelength to excite an administered fluorophore present in biological sample 108. For example, and without limitation, an iFS probe may include administering 5-aminolevulinic acid (5-ALA), to a patient orally 3 hours prior to surgery, wherein the iFS probe excites 5-ALA.

Still referring to FIG. 1, device 100 comprises an emission fiber optic 116. As used in this disclosure an “emission fiber optic” is a transparent fiber that transports one or more wavelengths of light associated with an emission wavelength. For example, and without limitation, emission fiber optic 116 may transport a 628 nm wavelength along a transparent fiber. As a further non-limiting example, emission fiber optic 116 may transport an 825 nm wavelength along a transparent fiber. As a further non-limiting example, emission fiber optic 116 may transport a 350 nm wavelength along a transparent fiber. As a further non-limiting example, emission fiber optic 116 may transport a 510 nm wavelength along a transparent fiber. As a further non-limiting example, emission fiber optic 116 may transport a 530 nm wavelength along a transparent fiber. As a further non-limiting example, emission fiber optic 116 may transport an 800 nm wavelength along a transparent fiber. As a further non-limiting example, emission fiber optic 116 may transport an 860 nm wavelength along a transparent fiber. In an embodiment, and without limitation, emission fiber optic 116 may transport a wavelength existing in a range of wavelengths along a transparent fiber. For example, and without limitation emission fiber optic 116 may transport a wavelength existing within the range of wavelengths of 510 nm-530 nm. As a further non-limiting example, emission fiber optic 116 may transport a wavelength existing within the range of wavelengths of 800 nm-860 nm. Emission fiber optic 116 is configured to detect an intrinsic emission 120 of biological sample 108. As used in this disclosure an “intrinsic emission” is a wavelength of light emitted as a function of an excited fluorophore present in biological sample. In an embodiment, and without limitation, intrinsic emission 112 may include an emission of light as a function of an excited fluorophore returning from an excited state to a ground state as a function of emitting a photon. In an embodiment, and without limitation, intrinsic emission 120 may include a vibrational mode. As used in this disclosure a “vibrational mode” is a periodic motion of the atoms of a molecule relative to each other. For example, and without limitation, vibrational mode may denote one or more vibrational frequencies that correspond to wavenumbers. As a further non-limiting example, vibrational mode may denote one or more harmonic motions, anharmonic motions, vibrational transitions, and the like thereof.

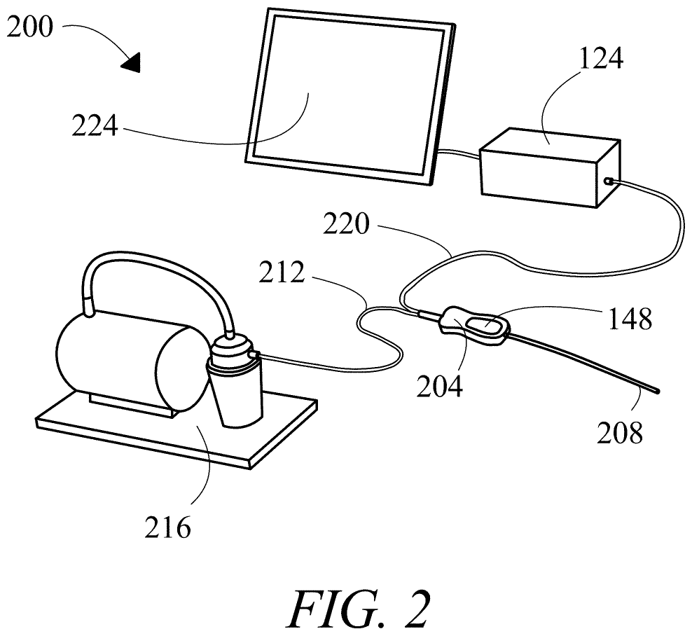

In an embodiment, and still referring to FIG. 1, emission fiber optic 116 may detect intrinsic emission 120 of biological sample 108 as a function of exciting biological sample 108 with a first wavelength. Emission fiber optic 116 may detect intrinsic emission 120 of biological sample 108 as a function of a second wavelength, wherein the first wavelength and the second wavelength are different and distinct. For example, and without limitation, excitation fiber optic 104 may excite a nervous tissue sample with a first wavelength of light comprising 525 nm, wherein emission fiber optic 116 may detect intrinsic emission 120 as a function of a second wavelength of light comprising 740 nm. In another embodiment, two sets of excitation wavelengths may be used, 405 nm and 635 nm, for excitation and detection of 5-ALA and/or PpIX. In an embodiment, and without limitation, excitation fiber optic 104 and emission fiber optic 116 may be extended passed the end of a tip of device 100, wherein a tip is described in detail below, in reference to FIG. 2. Additionally or alternatively, in an embodiment and without limit, a cancerous cell may preferentially produce and accumulate a natural, endogenous fluorophore, protoporphyrin IX (PpIX), due to uptake and metabolism of 5-ALA, causing the cancerous cell to provide an emission signal that differs from non-transformed cells. For example, and without limitation, intracellular PpIX is a substrate of the heme-synthesis pathway and emits red fluorescence (major peak of emission at 635 nm) when excited with blue light (405 nm). To achieve the appropriate light/photon wavelength necessary for excitation and emission of the light from 5-ALA and PpIX, excitation fiber optic 104 may supply a first wavelength, for instance and without limitation 405 nm light, and emission fiber optic 116 may detect a second wavelength, for instance and without limitation, 635 nm light.

Still referring to FIG. 1, device 100 comprises a tissue scanner 124. As used in this disclosure a “tissue scanner” is one or more devices and/or controllers that interprets and/or discerns an intrinsic emission. For example, and without limitation, tissue scanner 124 may include one or more detectors such as, charge-coupled devices (CCD), photodiodes, avalanche photodiodes (APDs), silicon photo-multipliers (SiPMs), complementary metal-oxide-semiconductor (CMOS), scientific CMOS (sCMOS), micro-channel plates (MCPs), micro-channel plate photomultiplier tubes (MCP-PMTs), single photon avalanche diode (SPAD), Electron Bombarded Active Pixel Sensor (EBAPS), quanta image sensor (QIS), spatial phase imagers (SPI), quantum dot cameras, image intensification tubes, photovoltaic imagers, optical flow sensors and/or imagers, photoresistors and/or photosensitive or photon-detecting circuit elements, semiconductors and/or transducers, and the like thereof. For instance, and without limitation, tissue scanner 124 may include a detector array, such as a detector array suitable for use as described above. Detectors in detector array may be sensitive specifically to a narrow band of wavelengths transmitted by light source, and/or may be sensitive to a range of wavelengths that includes the band transmitted by the light source. Detectors may be designed to react quickly to initial detection of photons, for instance through use of APDs or other highly sensitive detectors. Tissue scanner 124 is configured to discern a signal 128 representing intrinsic emission 120 of biological sample 120. As used in this disclosure a “signal” is one or more communications of information and/or data that is collected and/or transmitted. For example, and without limitation, signal may include one or more may include one or more wavelengths, vibrational frequencies, digital signals, analog signals, analog-to-digital conversions, binary signals, logic signals, and the like thereof. In an embodiment, and without limitation, tissue scanner 124 may be configured to determine a two-dimensional image and/or a three-dimensional image as a function of a multi-modal spectroscopy (MMS) scanner, wherein a multi-modal spectroscopy (MMS) scanner is described below. For example, and without limitation, MMS scanner may include one or more techniques such as but not limited to fluorescent spectroscopy, diffuse reflectance spectroscopy, Raman spectroscopy, optical coherence tomography, resonant ultrasound spectroscopy, and the like thereof to determine a two-dimensional and/or three-dimensional image.

In an embodiment, and still referring to FIG. 1, detectors may be on a focal plane that is offset the intrinsic emission of the biological sample, wherein tissue scanner 124 may include one or more one or more receptive optical elements, which may include collimating and/or focusing mirrors and/or lenses. One or more receptive optical elements may include filters such as without limitation dichroic, polarization, bandpass, notch, and/or other optical filters, which may act to screen out light that is not transmitted by light source; this may drastically increase signal to noise ratio. Tissue scanner 124 may include one or more optical elements that may include one or more reflective, diffractive, refractive, and/or metamaterial scanning elements for directing a beam from light source across a space to be scanned. As a non-limiting example, one or more optical elements may make use of a mirror galvanometer to direct a beam in scanning pattern. Scanning may be performed across two dimensions, using one or more optical elements and methods of directing individually or in combination for “beam steering,” including but not limited to, two flat or polygonal mirrors that may be driven by a galvanometer, electric motors, micro-electro machined systems (MEMS) or micro-optical electro machined systems (MOEMS) microscanner devices, piezoelectric actuated devices, magnetostrictive actuated devices, liquid, polymer, or other mechanically deformable devices, fast steering mirrors (FSM), Risley prisms, decentered macro-optical elements and micro-lens arrays, blazed grating optical elements, MOEMS or MEMS combined with macro-optical elements, phased arrays, electronically steered arrays, spatial light modulators (SLM), holographic optical elements, laser intra-cavity beam steering, and/or metamaterial surfaces or structures. A beam may alternatively or additionally be aimed and/or focused in three or more dimensions, for instance by using a servo-controlled lens system, which may be referred to without limitation as a “focus shifter,” “beam expander,” or “z-shifter.” Intensity of emitted light may alternatively or additionally be used. Mirrors perform a periodic motion using, for instance, rotating polygonal mirrors and/or a freely addressable motion, as in servo-controlled galvanometer scanners. Control of scanning motion may be affected via a rotary encoder and/or control electronics providing electric current to a motor or galvanometer controlling mirror angle. Electrical current may be varied using a servo controller digital to analog converter such as a DAC81416 as produced by Texas Instruments, Inc. of Dallas, Tex. Alternatively or additionally, the beam may be aimed and/or focused using a “non-mechanical” beam steering method, such as spatial light modulators (SLM) by adjusting the liquid crystal matrix that makes up the pixels of such device using digital or analog drive controllers to modify the angles of alignment of the liquid crystals as to make dynamic diffractive patterns to provide beam shaping and aiming. A laser's wavefront passing through the liquid crystal matrix is affected by the calculated diffractive patterns to provide both deflection of the beam for aiming, and an optical function for focusing or shaping the profile of the beam.

Still referring to FIG. 1, device 100 comprises a tissue scanner module 132. As used in this disclosure a “tissue scanner module” is a module and/or component that arranges and/or displays the intrinsic emission in a visualizable format. In an embodiment, and without limitation, tissue scanner module 132 may include a computing device. Computing device may include any computing device as described in this disclosure, including without limitation a microcontroller, microprocessor, digital signal processor (DSP) and/or system on a chip (SoC) as described in this disclosure. Computing device may include, be included in, and/or communicate with a mobile device such as a mobile telephone or smartphone. Computing device may include a single computing device operating independently, or may include two or more computing device operating in concert, in parallel, sequentially or the like; two or more computing devices may be included together in a single computing device or in two or more computing devices. Computing device may interface or communicate with one or more additional devices as described below in further detail via a network interface device. Network interface device may be utilized for connecting [computing device] to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. Computing device may include but is not limited to, for example, a computing device or cluster of computing devices in a first location and a second computing device or cluster of computing devices in a second location. Computing device may include one or more computing devices dedicated to data storage, security, distribution of traffic for load balancing, and the like. Computing device may distribute one or more computing tasks as described below across a plurality of computing devices of computing device, which may operate in parallel, in series, redundantly, or in any other manner used for distribution of tasks or memory between computing devices. Computing device may be implemented using a “shared nothing” architecture in which data is cached at the worker, in an embodiment, this may enable scalability of device 100 and/or computing device.

With continued reference to FIG. 1, computing device may be designed and/or configured to perform any method, method step, or sequence of method steps in any embodiment described in this disclosure, in any order and with any degree of repetition. For instance, computing device may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Computing device may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Still referring to FIG. 1, tissue scanner module 132 includes a display window 136. As used in this disclosure a “display window” is a portion of a display of tissue scanner module 132 and/or computing device to visualize intrinsic emission 120. Display window may include any display such as a light emitting diode (LED) screen, liquid crystal display (LCD), organic LED, cathode ray tube (CRT), touch screen, or any combination thereof. Display window may be configured to output intrinsic emission as a function of a graphical user interface and/or pictorial representation. Display window 136 may be configurable using executables, scripting languages, markup languages, and the like, including without limitation HTML, extensible stylesheet language transformations (XSLT), JavaScript, applets, and the like. In an embodiment, and without limitation, display window 136 may be incorporated into one or more head mounted displays, head-up displays, displays incorporated in eyeglasses, googles, headsets, helmet display systems, or the like, display window 136 may be incorporated in contact lenses, an eye tap display system including without limitation a laser eye tap device, VRD, or the like. In another embodiment, display window 136 may be incorporated into a stereoscopic display. As used in this disclosure a “stereoscopic display” is a display that simulates a user experience of viewing a three-dimensional space and/or object, for instance by simulating and/or replicating different perspectives of a user's two eyes; this is in contrast to a two-dimensional image, in which images presented to each eye are substantially identical, such as may occur when viewing a flat screen display window. Stereoscopic display may display two flat images having different perspectives, each to only one eye, which may simulate the appearance of an object or space as seen from the perspective of that eye. Alternatively or additionally, stereoscopic display may include a three-dimensional display such as a holographic display or the like. Persons skilled in the art, upon reviewing the entirety of this disclosure, will be aware of various alternative or additional types of stereoscopic display that may be employed in device 100.

Still referring to FIG. 1, display window 136 may be a window that ordinarily displays content when tissue scanner module 136 detects one or more intrinsic emissions 120. Additionally or alternatively, tissue scanner module 132 is configured to visualize intrinsic emission 120 of biological sample 108, wherein visualizing comprises displaying one or more representations on display window such as but not limited to, graphs, charts, images, pictures, data, videos, and the like thereof. Tissue scanner module 132 visualizes intrinsic emission 120 as a function of receiving signal 128 from tissue scanner 124 representing intrinsic emission 120 of biological sample 108. In an embodiment, and without limitation, tissue scanner module 132 may visualize intrinsic emission 120 as a function of mounting tissue scanner 124 within a vacuum-line tip, wherein a vacuum-line tip is described in detail below. For example, and without limitation, tissue scanner 124 may be mounted in the vacuum-line tip to at least generate a topographical and/or 3D rendering of biological sample 108. In an embodiment, and without limitation, 3D rendering may include a solid model of biological sample 108. As used in this disclosure a “solid model” is a computer model of three-dimensional solids. For example, and without limitation, solid model may include one or more geometric and/or solid models of biological sample 108. In an embodiment, and without limitation solid model may include a solid representation scheme such as a primitive instancing, spatial occupancy enumeration, cell decomposition, boundary representation, surface mesh modeling, sweeping, constructive solid geometry, implicit representation, parametric and/or feature-based modeling, and the like thereof. In an embodiment, and without limitation, solid model may incorporate one or more voxels, polygonal meshes, parametric shapes, and the like thereof to represent biological sample 108.

Still referring to FIG. 1, device tissue scanner module 132 is configured to relay a visual feed 140 as a function of signal 128 to display window 136. As used in this disclosure a “visual feed” is multimedia that is delivered and consumed in a continuous manner from a source. For example, and without limitation, visual feed 140 may include a live stream multimedia that at least represents one or more live and/or continuous multimedia feeds, recordings, and/or sources. In an embodiment, and without limitation, visual feed 140 may include one or more formats such as, but not limited to, digital video formats, digital audio formats, and the like thereof. In an embodiment, and without limitation, visual feed 140 may be relayed as a function of a wired communication. As a non-limiting example, wire communication may include relaying visual feed 140 as a function of a master bus controller, universal asynchronous receiver-transmitters (UART), universal serial bus (USBs), bus architecture, and the like thereof. In another embodiment, and without limitation, visual feed 140 may be relayed as a function of a wired communication. For example, and without limitation, wireless communication may include a communication using radio waves, electric fields, mobile broadband, Wi-Fi, and/or the BLUETOOTH protocol promulgated by Bluetooth SIG, Inc. of Kirkland, Wash., wherein Bluetooth is a wireless technology used for exchanging data between devices over short distances using ultra high frequency radio waves between 2.402 GHz to 2.480 GHz.

Still referring to FIG. 1, device 100 comprises a vacuum-line tip 144. As used in this disclosure a “vacuum-line tip” is a longitudinally extending element that comprises a tubular vacuum line within the tip, wherein a tip is described in detail below in reference to FIG. 2. For example, and without limitation, vacuum-line tip 144 may include one or more tips and/or extrusions comprising a tubular cavity that opens to the atmosphere to force particles, cells, tissues, and the like there of into the tip or extrusion as a function of a pressure difference. For example, and without limitation a pressure difference may include a difference of 400 Torr as a function of a tubular cavity pressure of 360 Torr and an environmental and/or ambient pressure of 760 Torr. Vacuum-line tip 144 is configured to remove a portion of biological sample 108 as a function of the visualized intrinsic emission 120 of biological sample 108. For example, and without limitation, vacuum-line tip may remove a cell, group of cells, tissue area, and the like thereof. As a further non-limiting example, vacuum-line tip may remove one or more cancerous cells, groups of cancerous cells, cancer tissue, and the like thereof. As a further non-limiting example vacuum-line tip may be configured to remove a bodily fluid such as blood, lymph fluid, cerebrospinal fluid, and the like thereof without removing one or more cells, tissues, and/or organs. In an embodiment, and without limitation, vacuum-line tip 144 may be configured to include a suction lumen, wherein a “suction lumen,” as used herein, is tubular cavity comprising a reduced pressure relative to the ambient pressure. In another embodiment, and without limitation, vacuum-line tip 144 may be configured to include a suction tip at the termination location of the vacuum-line tip, wherein a “suction tip,” as used herein, is a location of vacuum-line tip 144 that is at the termination location of the tip and interacts with biological sample 108. For example, and without limitation, suction tip may be a predetermined distance at the end of vacuum-line tip 124 to allow for excitation fiber optic 104 and/or emission fiber optic 116 to interact with biological sample 108. In an embodiment, and without limitation, removing the portion of biological sample 108 further comprises selecting a vacuum-powered force. As used in this disclosure a “vacuum-powered force” is a force exerted on an object as a function of a generated pressure differential. For example, and without limitation, vacuum-powered force may include a force exerted on a cell and/or tissue as a function of a pressure differential of 400 Torr due to a vacuum-line tip pressure of 260 Torr and an ambient pressure of 760 Torr. In an embodiment, and without limitation, vacuum-powered force may be selected as a function of an environmental parameter. As used in this disclosure an “environmental parameter” is a variable and/or parameter that effects the amount of force required to remove a portion of biological sample 108. In an embodiment, and without limitation, environmental parameter may include a plurality of parameters such as angle of vacuum, length of vacuum, depth of the portion of biological sample 108, surrounding light sources, and the like thereof. For example, and without limitation environmental parameter may denote that an external pressure is 758 Torr, wherein only 200 Torr is required to remove a portion of biological sample. As a further non-limiting example, environmental parameter may denote that a surrounding light source is reducing a resolutive capability of device 100, wherein vacuum-powered force should be reduced to prevent accidental biological sample 108 removal.

In an embodiment, and still referring to FIG. 1, vacuum-line tip 144 is configured to remove a portion of biological sample 108 as a function of the visualized intrinsic emission 120 of biological sample 108 and a haptic feedback controller 148. As used in this disclosure a “haptic feedback controller” is a device and/or component configured to alter and/or adjust a vacuum-powered force. In an embodiment, and without limitation, haptic feedback controller 148 may be a hole, thumbwheel, dial, slider, or any other suitable haptic feedback control for controlling vacuum-powered force. In an embodiment, and without limitation, vacuum tip, lumen, and/or haptic feedback controller 148 may be connected to a suction device. As used in this disclosure a “suction device” is device and/or component that voids a space and/or area of matter, as described below in detail. For example, and without limitation, suction device may include one or more positive displacement pumps, such as but not limited to rotary vane pumps, diaphragm pumps, liquid ring pumps, piston pumps, scroll pumps, screw pumps, Wankel pumps, external vane pumps, roots blowers, multistage roots pumps, Toepler pumps, lobe pumps, and the like thereof, momentum transfer pumps, regenerative pumps, entrapment pumps, venturi pumps, steam ejectors, and the like thereof. In an embodiment, and without limitation, haptic feedback controller 148 may include an adjustable vacuum switch and/or adjustable aperture to restrict and/or alter the amount of flow towards a suction device. For example, and without limitation, adjustable aperture may be reduced in diameter and/or radius to at least restrict the magnitude of vacuum-powered force generated as a function of reduced flow of pressure across the aperture. Alternatively or additionally, vacuum tip, lumen, and/or haptic feedback controller 148 may be connected to a vacuum line, such as a tube connected to a centralized or distributed vacuum system in a health care facility such as a hospital.

Referring now to the drawings, in FIG. 2, an exemplary embodiment 200 of a device 100 for intraoperative cancer detection is illustrated. In embodiments, device 100 may include a hand-held intrinsic fluorescence spectroscopy (iFS) controller 204. As used in this disclosure a hand-held intrinsic fluorescence spectroscopy (iFS) controller” is a controller to initiate and/or operate one or more fluorescence spectroscopy aspects of device 100. In an embodiment, and without limitation, hand-held iFS controller 204 may include a tip 208. As used in this disclosure a “tip” is a longitudinally extending component that protrudes from hand-held iFS controller 204. For example, and without limitation, tip 208 may include a protrusion comprising a distance of 0.3 meters. In an embodiment, and without limitation, hand-held iFS controller 204 may be secured and/or connected to a vacuum-line 212. As used in this disclosure a “vacuum-line” is a tubular structure and/or component comprising a tubular cavity with a lower pressure relative to the exterior of the structure. For example, and without limitation, vacuum-line 212 may include a tubular structure wherein the tubular cavity has a pressure of 300 Torr, wherein the exterior pressure of vacuum-line is 660 Torr. In an embodiment, and without limitation, vacuum-line 212 may be secured and/or attached to a suction device 216. As used in this disclosure a “suction device” is device and/or component that voids a space and/or area of matter. For example, and without limitation, suction device 216 may include one or more positive displacement pumps, such as but not limited to rotary vane pumps, diaphragm pumps, liquid ring pumps, piston pumps, scroll pumps, screw pumps, Wankel pumps, external vane pumps, roots blowers, multistage roots pumps, Toepler pumps, lobe pumps, and the like thereof, momentum transfer pumps, regenerative pumps, entrapment pumps, venturi pumps, steam ejectors, and the like thereof. In an embodiment, and without limitation, vacuum line 212 may removing a portion of biological sample 108 using a vacuum-powered force, wherein a vacuum-powered force is described in detail above in reference to FIG. 1. In an embodiment, and without limitation, hand-held iFS controller 204 may control and/or regulate vacuum-powered force as a function of haptic feedback controller 148, wherein haptic feedback controller 148 includes any of the haptic feedback controller 148 as described above, in reference to FIG. 1. As non-limiting example, haptic feedback controller 148 may include a hole by which a user may cover to supply suction through tip 208 or uncover to decrease or cease suction through tip 208. Haptic feedback controller 148 may include a thumbwheel, dial, slider, and/or any other suitable haptic feedback control for controlling suction power through tip 208.

In another embodiment, and without limitation, hand-held iFS controller 204 may be secured and/or connected to a fiber optics line 220. As used in this disclosure a “fiber optics line” is a connection and/or component that houses excitation fiber optic 104 and/or emission fiber optic 116 to allow for communication from iFS device 204 and tissue scanner module 132. For example, and without limitation, fiber optics line 220 may include a plurality of excitation fiber optics 104 and/or a plurality of emission fiber optics 116. In an embodiment, and without limitation, fiber optics line 220 may allow for excitation fiber optic 104 and/or emission fiber optic 116 to transcend through hand-held iFS device and terminate at the end of tip 208. In an embodiment, and without limitation fiber optics line 220 may be configured to supply light at one or more wavelengths for excitation and detecting emission of fluorophores, as described in further detail above, in reference to FIG. 1. In an embodiment, and without limitation, fiber optics line 220 may be allow for communication between handheld iFS device 204 and tissue scanner module 132, wherein tissue scanner module 132 is described above, in detail in reference to FIG. 1. In an embodiment, hand-held iFS device 204 may transmit the emission signal from a biological sample to tissue scanner module 132, wherein tissue scanner module 132 displays the visual feed on display window 136, wherein display window 136 is described above, in detail in reference to FIG. 1. In embodiments, tissue scanner module 132 can include digital and/or analog circuitry configured to receive, condition, and/or combine signals. In non-limiting exemplary embodiments, tissue scanner module 124 may be a multi-modal spectroscopy (MMS) scanner, which provides detailed information to assess the biological tissue emission more comprehensively and enhances the margin detection capability. MMS may be a combination of Raman spectroscopy, diffuse reflectance spectroscopy, iFS, and the like thereof. As used in this disclosure “Raman spectroscopy” is a spectroscopic technique to determine vibrational modes of molecules. For example, and without limitation, Raman spectroscopy may rely upon inelastic scattering of photons such as, but not limited to, Rayleigh scattering, Stokes Raman scattering, Anti-Stokes Raman scattering, and the like thereof. In an embodiment, and without limitation, Raman spectroscopy, may monitor and/or detect rotational and/or vibrational states of a tissue. As used in this disclosure a “diffuse reflectance spectroscopy” is a spectroscopic technique to determine a remission of a tissue. For example, and without limitation, diffuse reflectance spectroscopy may detect one or more reflections and/or back-scatterings of light and/or photons by a tissue. As a further non-limiting example, and without limitation, diffuse reflectance spectroscopy may determine and/or identify specular and/or diffuse back-scattered light. In an embodiment, and without limitation, tissue scanner module 124 may include one or more MMS scanners to assess the biological tissue emission. In non-limiting illustrative examples, tissue scanner module 124 may transmit one or more signals to display window 136; alternatively or additionally, tissue scanner module 124 feed may be displayed using a computing device, virtual reality (VR) and/or augmented reality (AR) headset of operating personnel.

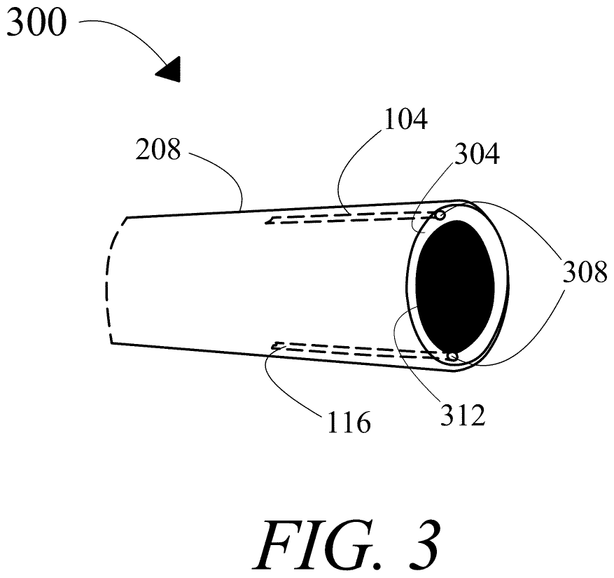

Referring now to FIG. 3, an exemplary embodiment, of an isometric cross-section view illustrating tip 208 is illustrated. Tip 208 may include a shape having one or more sides. Each side of tip 208 may comprise opposite, opposing surfaces with a thickness between them. In embodiments, a side of tip 208 may comprise a first surface forming a least a portion of an interior of tip 208 and a second opposite, opposing surface forming at least a portion of the exterior of tip 208. In embodiments, each side of tip 208 may form a wall 304 of tip 208 with each wall 304 comprising a first surface forming an interior of the wall and a second opposite, opposing surface forming an exterior of the wall 340.

With continued reference to FIG. 3, tip 208 may contain a fiber optic light 308 embedded or otherwise disposed within wall 304 of tip 208. As used in this disclosure a “fiber optic light” is a component that emits a wavelength of light. For example, and without limitation, fiber optic light 304 may include emitting one or more wavelengths such as gamma rays, x-rays, ultraviolet rays, visible light, infrared rays, microwaves, radio waves, and the like thereof. In a non-limiting example, tip 208 may contain a fiber optic light 308 for emitting and/or detecting an intrinsic fluorescence signal. For example, and without limitation, fiber optic light 308 may include an excitation fiber optic light and/or an emission fiber optic light. Here, a person of ordinary skill in the art will appreciate that a signal may be improved by the addition of a plurality of fiber optic lights 308 embedded within the walls 304 of tip 208. For instance, in non-limiting exemplary embodiments, 8 fiber optic lights may be embedded within the walls 304 of tip 208 to further increase the fluorescence signal during a surgery.

With continued reference to FIG. 3 fiber optic light 308 may include excitation fiber optic 104, wherein excitation fiber optic 104 includes any of the excitation fiber optic 104 as described above, in reference to FIG. 1. Excitation fiber optic 104 may include one or more optical fibers configured to supply light at a predetermined wavelength in order to stimulate emission from fluorophores adsorbed in the biological sample. Excitation fiber optic 104 may include one or more optical fibers configured to supply light at a predetermined wavelength in order to stimulate intrinsic emission from the biological sample. Fiber optic light 308 may comprise an emission fiber optic 116, wherein emission fiber optic 116 may supply light to stimulate emission from fluorophores adsorbed in the biological sample. Fiber optic light 308 may include an emission fiber optic 116, wherein emission fiber optic 116 may supply light to stimulate emission from aromatic residues in biological sample 108. Here, a person of ordinary skill in the art will understand that the fiber optics may be flush with the end of tip 208 in some embodiments and that the tube of tip 208 may extend past the termination of the fiber optics in other embodiments. One of ordinary skill in the art would understand that the placement of the end of the fiber optics may affect the focus of the light on the sample (i.e. the focal point of the fiber optics). Additionally or alternatively, tip 208 may include a tissue scanner 124, wherein a tissue scanner 124 is described above, in detail, in reference to FIG. 1.

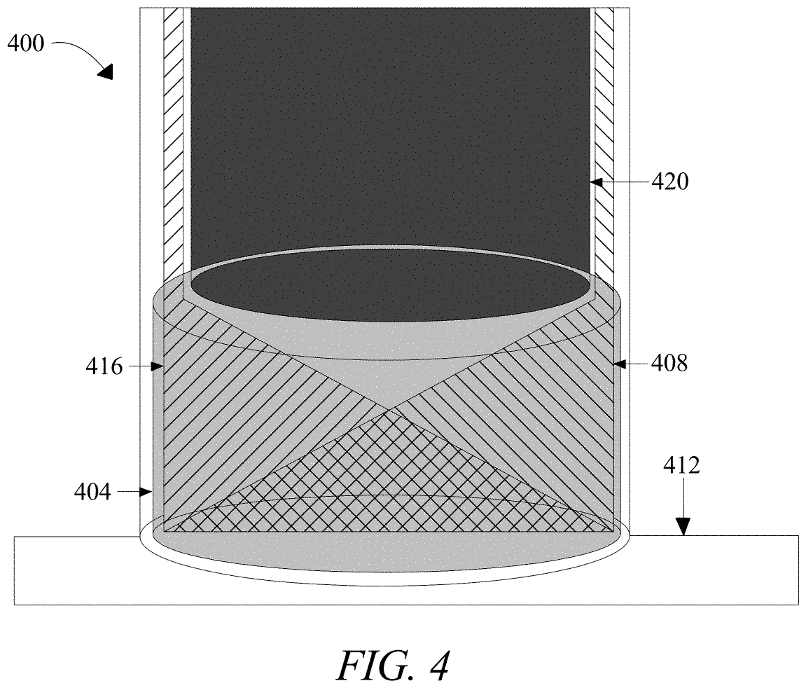

Now referring to FIG. 4, an exemplary embodiment 400 of suction tip 404 is illustrated, wherein suction tip 404 includes any of the suction tip as described above, in reference to FIGS. 1-3. Suction tip 404 may include an excitation signal 408. As used in this disclosure an “excitation signal” is a wavelength of light associated with an excitation of a fluorophore. For example, and without limitation, excitation signal 408 may include a 480 nm light that excites 50% of the fluorophores in biological sample 108. As a further non-limiting example, excitation signal 404 may include a 520 nm light that excites 80% of the fluorophores in biological sample 108. In an embodiment, excitation signal 408 may be angled, refracted, and/or reflected such that the signal interacts with a biological tissue 408 that tip 208 contacts. As used in this disclosure a “biological tissue” is a collection of interconnected cells that perform a similar function within an organism. For example, and without limitation, biological tissue 412 may include nervous tissue, brain tissue, neural networks, and the like thereof. Suction tip may include an emission signal 416. As used in this disclosure an “emission signal” is a wavelength of light associated with an emission of a fluorophore. For example, and without limitation, emission signal 416 may be received as a function of biological sample 108 emitting a wavelength as a function of a fluorophore emission. As a further non-limiting example, emission signal 416 may be received as a function of biological sample 108 emitting a wavelength as a function of an aromatic residue emission. In an embodiment, emission signal may be angled, refracted, and/or reflected such that the signal contacts and/or stimulates emission fiber optic 116. In an embodiment, and without limitation suction tip 404 may be adjacent to a suction lumen 420. Suction lumen 420 includes any of the suction lumen 420 as described above, in reference to FIGS. 1-3.

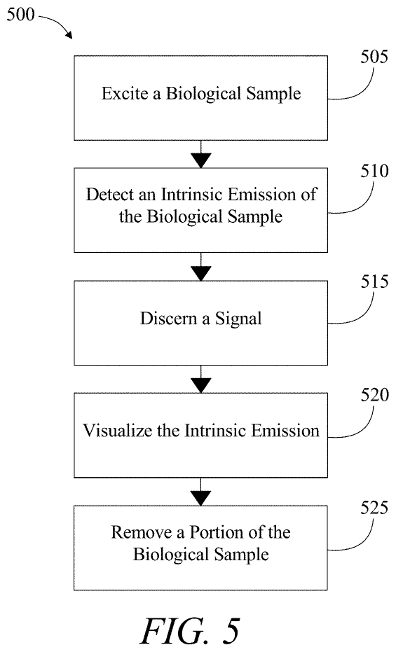

Referring now to FIG. 5, a method 500 for intraoperative cancer detection is illustrated. At step 505, device 100 excites, as a function of an excitation fiber optic 104, a biological sample 108 as a function of an intrinsic excitation wavelength 112. Excitation fiber optic 104 includes any of the excitation fiber optic as described above, in reference to FIGS. 1-4. Biological sample 108 includes any of the biological sample 108 as described above, in reference to FIGS. 1-4. Intrinsic excitation wavelength 112 includes any of the intrinsic excitation wavelength 112 as described above, in reference to FIGS. 1-4. In an embodiment, and without limitation, excitation of cells may be performed using an intrinsic fluorescence spectroscopy probe which may include administering a fluorophore to a biological sample; this may be implemented, without limitation, as described above in FIG. 1-4.

Still referring to FIG. 5, at step 510, device 100 detects, as a function of an emission fiber optic 116, an intrinsic emission 120 of biological sample 108. Emission fiber optic 116 includes any of the emission fiber optic 116 as described above, in reference to FIGS. 1-4. Intrinsic emission 120 includes any of the intrinsic emission 120 as described above, in reference to FIGS. 1-4. In an embodiment, and without limitation, detecting intrinsic emission 120 of biological sample 108 may include exciting biological sample 108 with a first wavelength and detecting intrinsic emission 120 of biological sample 108 as a function of a second wavelength, wherein the first wavelength and the second wavelength are distinct; this may be implemented, without limitation, as described above in FIG. 1-4.

Still referring to FIG. 5, at step 515, device 100 discerns a signal 128 as a function of a tissue scanner 124. Signal 128 includes any of the signal 128 as described above, in reference to FIGS. 1-4. Tissue scanner 124 includes any of the tissue scanner 124 as described above, in reference to FIGS. 1-4.

Still referring to FIG. 5, at step 520, device 100 visualizes, as a function of a tissue scanner module 132 including a display window 136, intrinsic emission 120 of biological sample 108. Tissue scanner module 132 includes any of the tissue scanner module 132 as described above, in reference to FIGS. 1-4. Display window 136 includes any of the display window 136 as described above, in reference to FIGS. 1-4. Device 100 visualizes intrinsic emission 120 of biological sample 108 as a function of receiving signal 128 from tissue scanner 124. Device 100 visualizes intrinsic emission 120 of biological sample 108 by relaying a visual feed 140 as a function of signal 128 to display window 136. Visual feed 140 includes any of the visual feed 140 as described above, in reference to FIGS. 1-4. In an embodiment, and without limitation, visualizing may include using a camera mounted on the tip of the fluorescence spectroscopy device that can relay visual recording of the emission profile of a tissue. Visualizing the emission signal from a biological sample may include mounting a tissue scanner within the tip of the hand-held fluorescence spectroscopy device; this may be implemented, without limitation, as described above in FIG. 1-4.

Still referring to FIG. 5, at step 525, device 100 removes, as a function of a vacuum-line tip 144, a portion of biological sample 108 as a function of the visualized intrinsic emission 120 of biological sample 108 and a haptic feedback controller 148. Vacuum-line tip 144 includes any of the vacuum-line tip 144 as described above, in reference to FIGS. 1-4. Haptic feedback controller 148 includes any of the haptic feedback controller 148 as described above, in reference to FIGS. 1-4.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

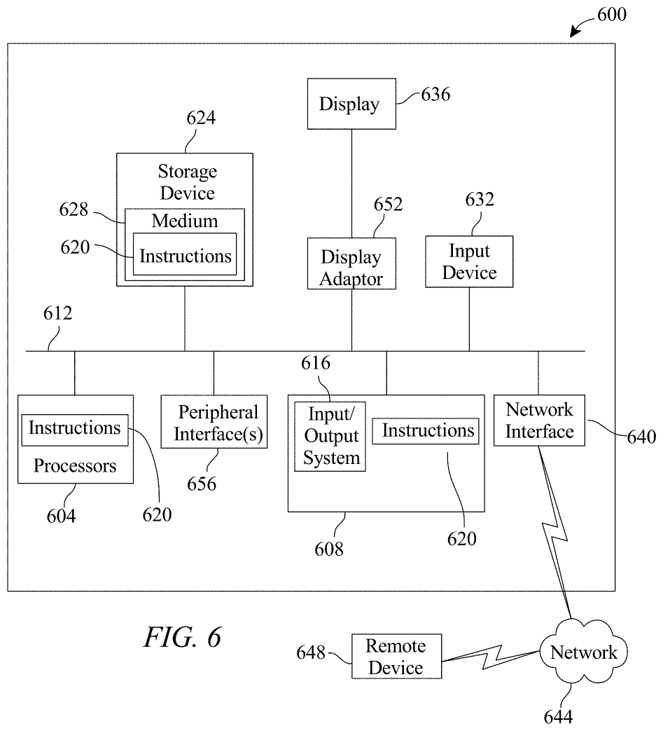

FIG. 6 shows a diagrammatic representation of one embodiment of a computing device in the exemplary form of a computer system 600 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 600 includes a processor 604 and a memory 608 that communicate with each other, and with other components, via a bus 612. Bus 612 may include any of several types of bus structures including, but not limited to, a memory bus, a memory controller, a peripheral bus, a local bus, and any combinations thereof, using any of a variety of bus architectures.

Processor 604 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 604 may be organized according to Von Neumann and/or Harvard architecture as a non-limiting example. Processor 604 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), and/or system on a chip (SoC).

Memory 608 may include various components (e.g., machine-readable media) including, but not limited to, a random-access memory component, a read only component, and any combinations thereof. In one example, a basic input/output system 616 (BIOS), including basic routines that help to transfer information between elements within computer system 600, such as during start-up, may be stored in memory 608. Memory 608 may also include (e.g., stored on one or more machine-readable media) instructions (e.g., software) 620 embodying any one or more of the aspects and/or methodologies of the present disclosure. In another example, memory 608 may further include any number of program modules including, but not limited to, an operating system, one or more application programs, other program modules, program data, and any combinations thereof.

Computer system 600 may also include a storage device 624. Examples of a storage device (e.g., storage device 624) include, but are not limited to, a hard disk drive, a magnetic disk drive, an optical disc drive in combination with an optical medium, a solid-state memory device, and any combinations thereof. Storage device 624 may be connected to bus 612 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 624 (or one or more components thereof) may be removably interfaced with computer system 600 (e.g., via an external port connector (not shown)). Particularly, storage device 624 and an associated machine-readable medium 628 may provide nonvolatile and/or volatile storage of machine-readable instructions, data structures, program modules, and/or other data for computer system 600. In one example, software 620 may reside, completely or partially, within machine-readable medium 628. In another example, software 620 may reside, completely or partially, within processor 604.

Computer system 600 may also include an input device 632. In one example, a user of computer system 600 may enter commands and/or other information into computer system 600 via input device 632. Examples of an input device 632 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 632 may be interfaced to bus 612 via any of a variety of interfaces (not shown) including, but not limited to, a serial interface, a parallel interface, a game port, a USB interface, a FIREWIRE interface, a direct interface to bus 612, and any combinations thereof. Input device 632 may include a touch screen interface that may be a part of or separate from display 636, discussed further below. Input device 632 may be utilized as a user selection device for selecting one or more graphical representations in a graphical interface as described above.

A user may also input commands and/or other information to computer system 600 via storage device 624 (e.g., a removable disk drive, a flash drive, etc.) and/or network interface device 640. A network interface device, such as network interface device 640, may be utilized for connecting computer system 600 to one or more of a variety of networks, such as network 644, and one or more remote devices 648 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 644, may employ a wired and/or a wireless mode of communication. In general, any network topology may be used. Information (e.g., data, software 620, etc.) may be communicated to and/or from computer system 600 via network interface device 640.

Computer system 600 may further include a video display adapter 652 for communicating a displayable image to a display device, such as display device 636. Examples of a display device include, but are not limited to, a liquid crystal display (LCD), a cathode ray tube (CRT), a plasma display, a light emitting diode (LED) display, and any combinations thereof. Display adapter 652 and display device 636 may be utilized in combination with processor 604 to provide graphical representations of aspects of the present disclosure. In addition to a display device, computer system 600 may include one or more other peripheral output devices including, but not limited to, an audio speaker, a printer, and any combinations thereof. Such peripheral output devices may be connected to bus 612 via a peripheral interface 656. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve devices and methods according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

What is claimed is:

1. A device for intraoperative cancer detection, the device comprising:
an excitation fiber optic configured to excite a biological sample as a function of an intrinsic excitation wavelength;
an emission fiber optic configured to detect an intrinsic emission of the biological sample;
a tissue scanner configured to discern a signal representing the intrinsic emission of the biological sample;
a tissue scanner module including a display window configured to visualize the intrinsic emission of the biological sample, wherein visualizing further comprises:
receiving the signal from the tissue scanner; and
relaying a visual feed as a function of the signal to the display window; and
a vacuum-line tip configured to remove a portion of the biological sample as a function of the visualized intrinsic emission of the biological sample and a haptic feedback controller.

2. The device of claim 1, wherein exciting the biological sample further comprises utilizing an intrinsic fluorescence probe excited by an excitation wavelength.

3. The device of claim 1, wherein exciting the biological sample further comprises administering an intrinsic fluorescence spectroscopy fluorophore to the biological sample.

4. The device of claim 3, wherein the intrinsic fluorescence spectroscopy fluorophore is 5-aminolevulinic acid.

5. The device of claim 1, wherein detecting the intrinsic emission of the biological sample further comprises:
exciting the biological sample with a first wavelength; and
detecting the intrinsic emission of the biological sample as a function of a second wavelength, wherein the first wavelength and the second wavelength are distinct.

6. The device of claim 1, wherein the intrinsic emission of the biological sample includes a vibrational mode of the biological sample.

7. The device of claim 1, wherein visualizing the intrinsic emission of the biological sample further comprises mounting the tissue scanner within the vacuum-line tip.

8. The device of claim 1, wherein removing the portion of the biological sample further comprises selecting a vacuum-powered force.

9. The device of claim 8, wherein the vacuum-powered force is selected as a function of an environmental parameter.

10. The device of claim 1, wherein the haptic feedback controller connected to a suction device.

11. A hand-held fluorescence spectroscopy method for intraoperative cancer detection, the method comprising:
exciting, as a function of an excitation fiber optic, a biological sample as a function of an intrinsic excitation wavelength;
detecting, as a function of an emission fiber optic, an intrinsic emission of the biological sample;
discerning, as a function of a tissue scanner, a signal representing the intrinsic emission of the biological sample;
visualizing, as a function of a tissue scanner module including a display window, the intrinsic emission of the biological sample, wherein visualizing further comprises:
receiving the signal from the tissue scanner; and
relaying a visual feed as a function of the signal to the display window; and
removing, as a function of a vacuum-line tip, a portion of the biological sample as a function of the visualized intrinsic emission of the biological sample and a haptic feedback controller.

12. The method of claim 11, wherein exciting the biological sample further comprises utilizing an intrinsic fluorescence probe excited by an excitation wavelength.

13. The method of claim 11, wherein exciting the biological sample further comprises administering an intrinsic fluorescence spectroscopy fluorophore to the biological sample.

14. The method of claim 13, wherein the intrinsic fluorescence spectroscopy fluorophore is 5-aminolevulinic acid.

15. The method of claim 11, wherein detecting the intrinsic emission of the biological sample further comprises:
exciting the biological sample with a first wavelength; and
detecting the intrinsic emission of the biological sample as a function of a second wavelength, wherein the first wavelength and the second wavelength are distinct.

16. The method of claim 11, wherein detecting the intrinsic emission of the biological sample further comprises identifying an intrinsic infrared emission of the biological sample.

17. The method of claim 11, wherein visualizing the intrinsic emission of the biological sample further comprises mounting the tissue scanner within the vacuum-line tip.

18. The method of claim 11, wherein removing the portion of the biological sample further comprises selecting a vacuum-powered force.

19. The method of claim 18, wherein the vacuum-powered force is selected as a function of an environmental parameter.

20. The method of claim 11, wherein the haptic feedback controller connected to a suction device.
